(19) United States
(12) Patent Application Publication
(10) Pub. No.: US 2017/0152296 A1
(43) Pub. Date: Jun. 1, 2017

(54) COMPOSITIONS FOR THE TREATMENT OF WOUNDS

(71) Applicant: Wei Li, Los Angeles, CA (US)

(72) Inventor: Wei Li, Los Angeles, CA (US)

(21) Appl. No.: 15/365,908

(22) Filed: Nov. 30, 2016

Related U.S. Application Data
(60) Provisional application No. 62/261,796, filed on Dec. 1, 2015.

(51) Int. Cl. C07K 14/47; A61K 45/06; A61K 9/00; A61K 38/17; A61K 38/18

(52) U.S. Cl. CPC C07K 14/4705; A61K 38/1709; A61K 38/1858; A61K 9/0014; A61K 45/06; C07K 2319/00

(57) ABSTRACT

Recombinant polypeptides and compositions as described herein and are useful in methods to promote epidermal tissue regeneration and/or to promote wound healing in a variety of tissues subject in need thereof. The method comprises administering to a tissue or wound in need thereof an effective amount of a recombinant polypeptide operatively linked to a carrier protein or a composition containing the recombinant polypeptide linked to a carrier protein.

### CROSS-REFERENCE TO RELATED APPLICATION

This application claims priority under 35 U.S.C. §119(e) to U.S. Provisional Application No.62/261,796, filed Dec. 1, 2015, the content of which is hereby incorporated by reference in its entirety.

### TECHNICAL FIELD

This disclosure resides in the field of wound healing compositions and use thereof. Particularly, this disclosure relates to compositions of polypeptides and the topical application of these positions to the skin to expedite wound healing by promoting all skin cell migration, especially keratinocytes, dermal fibroblasts and dermal microvascular endothelial cells.

### BACKGROUND

Throughout this disclosure, various publications, patents and published patent specifications are referenced by an identifying citation or an Arabic number. The full citations for the references identified by an Arabic number are found in this disclosure, immediately preceding the claims. The disclosures of these publications, patents and published patent specifications are hereby incorporated by reference into the present disclosure in their entirety to more fully describe the state of the art to which this invention pertains.

Rodents such as rats and mice have been the widely used animals for skin wound healing studies. However, these models are less than ideal because they are loose skinned animals and the way they heal skin wounds is predominantly by the mechanism of wound contraction, which may not translate well to human skin wound healing. Pigs, like human beings, are tight skinned animals and heal skin wounds with a larger component of re-epithelialization (i.e., the lateral migration of keratinocytes across the wound bed) and a smaller component of wound contraction. Moreover, pigs are also effective models for topical medication studies, because multiple groups of replicate wounds can be created in the same pig for studies of comparative agents. In randomized wound healing studies, for instance, there is a high concordance of the results between pigs and humans [1-4].

Current literature on pig wound healing models shows that few of those previous studies made efforts to first standardize the critical parameters, such as the relationship between locations of wound and their healing rates, optimal distance between two wounds, measurements of diabetic markers over time, correlation between diabetic conditions and delay in wound closure, just to mention a few, prior to using the animals to carry out wound healing studies. There is a need to re-evaluate these parameters and provide established methods for using pigs as wound healing models [1].

### SUMMARY

At the forefront of wound healing therapeutics, growth factors are thought to serve as the driving force of wound healing and, therefore, have been the focus for therapeutic development of wound healing agents [5]. After decades of investigations and clinical trials, however, the human recombinant platelet-derived growth factor (PDGF) remains the only FDA-approved growth factor for the topical treatment of human diabetic ulcers. This therapy, becaplermin gel (Regranex™), has since been shown by multi-center, double blinded and randomized clinical studies to have a modest efficacy. In addition, it showed a fivefold higher potential of causing cancer in patients. Applicant's recent studies identified three molecular hurdles against conventional growth factors and these hurdles could significantly reduce the effectiveness of PDGF-BB/becaplermin gel.

First, PDGF-BB only affects dermal fibroblasts, due to the lack of PDGF receptors in two other skin cell types, human keratinocytes and human dermal microvascular endothelial cells. Thus, conventional growth factors simply cannot fulfill the task of promoting wound closure during the critical early phase of wound healing—re-epithelialization.

Second, PDGF-BB-stimulated cell proliferation and migration are completely blocked by the TGFβ family of cytokines, which are abundant in the wound bed. Wound healing, or wound repair, is an intricate process in which the skin repairs itself after injury. In normal skin, the epidermis (outermost layer) and dermis (inner or deeper layer) exists in a steady-stated equilibrium, forming a protective barrier against the external environment. The normal wound healing process can be broadly classified into three stages namely the inflammatory, proliferative and maturation phases. The inflammatory phase lasts 0-2 days and involves an orderly recruitment of cells to the wound area. This is followed by the 2-6 day proliferative phase, in which fibroblasts, keratinocytes and other cells in the wound bed begin to actively proliferate to close the wound. During the first phase of tissue repair, an acute inflammatory response with cellular migration occurs. Neutrophils predominate for the first 24-48 hours; macrophages become active by the third day. The neutrophils and macrophages phagocytose and digest pathologic organisms and tissue debris. The maturation phase follows the proliferative phase, peaking at 21 days, by which time the wound is completely healed by restructuring the initial scar tissue. Third, PDGF-BB's biological effects are significantly compromised under the environment of hyperglycemia, the signature for diabetes of all types [6-8].

Other research has involved the use of heat shock protein to promote wound healing. For example, Srivastava et al. discloses in U.S. Pat. No. 6,475,490 compositions comprising heat shock proteins, including gp96, hsp90, and hsp70, uncomplexed or complexed noncovalently with antigenic molecules. However, the use of the entire length of these large molecules in true pharmaceutical compositions could cause higher immune responses/inflammation from the host and hit higher number of unnecessary off targets. In addition, it is more expensive to manufacture larger protein than its replacement of a smaller peptide.

The above-mentioned disappointing outcomes with conventional growth factors prompted Applicant to search for alternative molecules that could overcome the three obstacles mentioned previously. These efforts led to the discovery of the disclosed methods which exploit the discovery that secreted form of heat shock protein-90αalpha (Hsp90α), which is a novel pro-motility factor, is resistant to TGFβ and hyperglycemia and has its receptor expressed in every cell type. The topical application of recombinant Hsp90α promotes wound healing in both healthy and diabetic mice [8, 9]. Applicant also established and re-standardizes both acute and diabetic pig wound healing models, including wound size, surgical pattern, correlation between diabetic conditions and delay in wound healing to further establish the importance of Hsp90α in skin wound healing as a potential therapeutic for humans. This disclosure further provides evidence that only prolonged diabetic conditions are associated with a delay in diabetic wound healing. Then, with the use of these novel models, this disclosure identifies the minimum essential entity in the secreted form of the heat shock protein-90αalpha (Hsp90α), a 27-amino acid peptide, termed F-8 and its equivalents, as an optimal therapeutic entity.

In view of the above, in one aspect, the disclosure provides a systematic evaluation and establishment of both acute and diabetic wound healing models in pigs, including wound-creating pattern for drug treatment versus control, measurements of diabetic parameters and the time for detecting delayed wound healing in a diabetic pig model.

In one aspect, this disclosure also provides a recombinant polypeptide comprising, or alternatively consisting essentially of, or yet further consisting of, an Hsp90α polypeptide sequence (SEQ ID NO.1), an F-8 polypeptide sequence (SEQ ID NO. 2), an F-5 polypeptide sequence (SEQ ID NO. 3), an F-6 polypeptide sequence (SEQ ID NO. 4), or an equivalent thereof operatively linked to a non-immunogenic carrier protein, wherein the recombinant polypeptide is operatively linked to a non-immunogenic carrier protein. In one aspect, the recombinant polypeptide comprises, alternatively consists essentially of, or yet further consists of the 27-amino acid sequence of F-8 as a minimum amino acid sequence. In one aspect, the recombinant polypeptide comprises, alternatively consists essentially of, or yet further consists of additional amino acid sequence on either or both the amine or carboxy termini of the F-8 sequence. In one aspect, the additional amino acid sequence comprises, alternatively consists essentially of, or yet further consists of amino acids from the protein Hsp90α, e.g., the amino acid sequence that is within the polypeptide F-5 fragment consisting of 115 amino acid units which is from amino acid number 236 to 350 of Hsp90α, or alternatively the F-6 fragment having 54 amino acid units from amino acid number 236 to 289 of Hsp90α (SEQ ID NO. 4). In one aspect, the polypeptide is not a wild-type full length protein.

The recombinant polypeptides and compositions as described herein are useful in methods to promote epidermal tissue regeneration and/or re-epithelialization or prevent cell apoptosis in wounded epithelial tissue, in a subject in need thereof, the method comprising, or alternatively consisting essentially of, or yet further consisting of, administering to a tissue in need thereof an effective amount of the polypeptide or composition as disclosed herein, thereby promoting epidermal tissue regeneration, and/or re-epithelialization or prevent cell apoptosis in wounded epithelial tissue. In one aspect the subject is a mammal, e.g. a human patient.

The recombinant polypeptides and compositions as described herein also are useful in methods to promote wound healing, or to treat or heal wounds in a subject in need thereof, the method comprising, or alternatively consisting essentially of, or yet further consisting of, administering to the wounded tissue in need thereof an effective amount of the polypeptide or composition as disclosed herein. In one aspect the subject is a mammal, e.g. a human patient. In a further aspect, the wound is an acute wound. In another aspect, the wound is a burn wound, e.g., a secondary burn wound progression or conversion. In one aspect, the wound is a diabetic wound. Without being bound by a theory, the peptide designed F-5 has dual functions to promote burn wound healing. Burn wounds are different from other acute wounds, such as traumatic and surgical wounds. They expand from their initial boundary of trauma to larger areas horizontally and vertically. This burn wound-specific phenomenon is called secondary burn wound progression or conversion, in which death of the surrounding cells over time is a major pathophysiological factor. This disclosure provides that the peptide designated F-5 alone prevents burn wound progression through preventing cell apoptosis in the burned skin and thereafter promotes burn wound re-epithelialization (closure). Since the core activity of the 155-amino acid F-5 is determined by a 27-amino acid domain within F-5, identified herein as F-8, and considering the criteria for treating acute wounds (from healthy humans), i.e. high specificity, low off-target effect and less host immune response, the F-8 fragment with a carrier (to ensure stability in the hostile wound environment) is more useful than F-5 to achieve these therapeutic outcomes as well. This finding is especially encouraging for those who get burn injuries in the battle field, for which there currently are no drugs or therapies to treat these wounds.

The method optionally uses a pharmaceutical composition having a pharmaceutical medium to carry the polypeptide compound, consisting of an aqueous solution, suspension, dispersion, salve, ointment, gel, cream, lotion, spray or paste. In a further aspect, the formulation is a lyophilized “powder” for ease of transport.

In one aspect, the disclosure provides a method to promote epidermal tissue regeneration and/or re-epithelialization or prevent cell apoptosis in wounded epithelial tissue or in tissue of the eye, in a subject in need thereof comprising administering to a tissue in need thereof an effective amount of the recombinant polypeptide. In another aspect, the disclosure provides a method to facilitate healing or treat a wound or injury, comprising administering to a wounded or injured tissue in a subject in need thereof an effective amount of the recombinant polypeptide. In one embodiment, the method further comprises administering an effective amount of a wound-healing therapeutic agent other than the recombinant polypeptide. In another embodiment, the administration of the recombinant polypeptide and the therapeutic agent is concurrent or sequential. In one embodiment, the subject is a mammal.

In some embodiment, the wound is a skin wound or a wound to an eye. In one embodiment, the skin wound is an acute wound, a diabetic wound, or a burn wound. In some embodiments, the acute wound comprises a traumatic wound or a surgical wound. In another embodiment, the method of treating a wound further comprises treating or preventing progression or conversion of the burn wound.

In one aspect, the injury is an eye disease or an eye injury. In one embodiment, the eye injury is a corneal injury or a conjunctival injury. In another embodiment, the eye injury is caused by a penetrating object, a foreign body, a chemical, or burn.

In one embodiment of the method of wound healing, the composition is applied to the wound about every 3 to 72, or alternatively at least about every 6 to about every 72 hours. Optionally, the composition is applied to the wound about every 24 to about every 48 hours.

Additional advantages and other features of the present disclosure will be set forth in part in the description which follows and in part will become apparent to those having ordinary skill in the art upon examination of the following or may be learned from the practice of the disclosure. The advantages of the disclosure may be realized and obtained as particularly pointed out in the appended claims.

As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 shows comparison among human, pig, rat and mouse skin. Paraffin skin sections from various depths of tissue from healthy humans, pigs, rats and mice were simultaneously subjected to H&E staining for structural comparisons. Representative images from each of the four species are shown with the same magnification scale. EP, epidermis; DM, dermis; BV, blood vessel; SB, sebaceous gland, HF, hair follicle, AP, apocrine (sweat) gland; and M, muscle. The measurement bars are as indicated.

FIGS. 2A-2G show establishment of the wound pattern for control versus topical drug treatments. (FIGS. 2A, 2B and 2C) 2.0 cm 62.0 cm full thickness wounds were created on the same side of the pigs (n53) with 2.5 cm apart between wounds were compared either between the top and the bottom wounds (FIG. 2A) or between the middle and rear wounds (FIG. 2B) or between wounds made at similar spots, but on the opposite side of the pig (FIG. 2C). Quantitations (% of healing) were made based on triplicate wounds in each pig and shown below each of the images. (FIGS. 2D and 2E) A schematic presentation of nine 2.0 cm 62.0 cm (in normal pigs) or 1.5 cm×1.5 cm (in diabetic pigs) full thickness wounds were created on each side of the pig with 2.5 cm apart between wounds. Comparisons between treatment and control should be made between two wounds at similar spots, but on the opposite side of the pig, as indicated by color squares marked in the same color. (FIGS. 2F and 2G) Based on the above design, real wounds were created on the two sides of pigs. Treatments versus controls are indicated.

FIGS. 3A-3D show recombinant F-5 fragment of Hsp90α promoted wound healing in normal pigs. (FIG. 3A) Picture of a typical 25-30 kg and healthy farm pig used in experiments. (FIG. 3B) Wounds (2.0 cm×2.0 cm) in triplicates were topically treated once on day 0 with either CMC gel alone or CMC gel containing recombinant F-5 protein (45 mM). Wounds in triplicate were photographed on the days indicated and analyzed for wound closure rates. (FIG. 3C) Quantitative analyses of the wound closure were presented. (FIG. 3D) Wedge biopsies were made on day 14 wounds, sectioned and stained with H&E. This experiment was repeated four times (a single surgery conducted in four separate pigs) and the results were reproducible. * p #0.05, ** p #0.01 and *** p #0.001, compared with the placebo.

FIGS. 4A-4E show characteristics of diabetes and delayed wound healing in STZ-treated pigs. (FIG. 4A) A pig, 8 weeks after STZ injection and having undergone the first round of wound healing experiments, was having her breakfast. The pig weighed 36 kg on that day. (FIG. 4B) Sections of pancreas removed from normal and STZ-treated pigs were subjected to anti-insulin antibody immunohistochemistry staining. The results show complete disappearance of insulin-producing islets in STZ-treated pigs. (FIG. 4C) A typical four-month blood glucose profile of a pig following STZ injection showed hyperglycemia throughout the period of experiments. (FIG. 4D) For a typical weight profile of a STZ-treated pig, in comparison to normal pigs, 50 kg is the size limitation (red dotted line) of the facility, when pigs needed to be euthanized. (FIG. 4E) Elevated A1c levels in circulation were detected in three diabetic pigs, in comparison to three controls.

FIGS. 5A-5C show comparisons between normal and diabetic skin structures in humans and pigs. Comparisons between normal and diabetic skin structures in humans and pigs to investigate if there were similar changes in skin structure between STZ-induced diabetic pigs and humans with diabetes, we obtained skin from diabetic and non-diabetic humans and from normal and STZ-induced diabetic pigs and examined them by H&E histology and immunohistochemistry analyses. (FIG. 5A) H&E staining of normal human skin, normal pig skin, diabetic human skin and diabetic pig skin. The black dotted lines (panels b and d) illustrate the dense ECM structure of normal pig skin compared to the looser ECM structure of the diabetic pig skin. (FIG. 5B) Picrosirius Red staining visualized under polarized light illustrates collagen structure. The white arrows (panels g and h) illustrate the dark areas revealing less density in collagen fibers of the diabetic human and pig tissue compared to their normal counterparts (panels e and f). (FIG. 5C) AGE immunohistochemistry staining reveals less accumulation of Advanced Glycation End Products in the normal human skin compared to the diabetic skin (panel I vs. panel k), see blue arrows. A similar trend is seen in the normal pig skin compared to the diabetic pig skin (panel j vs. l).

FIGS. 6A-6G show duration of diabetic conditions correlated with the length of delay in wound healing. The length (days) of delay in wound healing was examined in pigs injected with STZ for 20, 45 and 90 days prior to wound surgery. (FIG. 6A) Images of the 1.5 cm 61.5 cm full thickness wounds made in a representative normal pig; (FIG. 6B) Images of the wound healing in a representative pig 20 days after STZ injection; (FIG. 6C) Images of wounds in a representative pig 45 days after STZ injection; (FIG. 6D) Images of wounds in a representative pig 90 days after STZ injection; (FIGS. 6E to 6G) Quantitative analyses of the wound healing data shown in FIGS. 6B, 6C and 6D (n>3) in comparison to wounds shown in A. * p #0.05, ** p #0.01 and *** p #0.001, compared with placebo.

FIGS. 7A-7D show comparison of F-5 with becaplermin gel in promoting diabetic pig wound healing. (FIG. 7A) Images of 1.5 cm×1.5 cm full thickness wounds in triplicates in pigs 45 days following STZ injection were topically treated with increasing amounts of recombinant F-5 protein once on day 0. Wound closure was measured on day 7 and day 14. (FIG. 7B) Quantitative analysis of the wound closure data revealed an optimal concentration for F-5 between 30 mM to 45 mM. * p #0.05 and ** p #0.01, compared with placebo. (FIG. 7C) Images of similar wounds were topically treated with 45 mM of F-5 protein or becaplermin gel as prescribed in triplicate on day 0. Wound closure was compared on day 7 and day 14. (FIG. 7D) Quantitative analysis of F-5- and becaplermin gel-stimulated wound closure data. * p #0.05, p #0.01 and *** p #0.001, compared with placebo.

FIGS. 8A-8F show histological analyses of healed wounds treated with F-5 versus becaplermin. Skin biopsies of CMC, F-5-treated and becaplermin-treated diabetic pig wounds on day 14 were subjected to various histochemistry and immunohistochemistry analyses. (FIG. 8A) H&E staining showed rete ridge production between the epidermis (green arrows) and dermis (panel b vs. panel a and panel c). Insert is pan keratin antibody staining showing the re-epithelialization tongue (red line and red arrows), the orange line shows the unhealed area devoid of epidermis. (FIG. 8B) Anti-PECAM-1 antibody staining indicated more blood vessel formation in the newly healed wound site of both F-5 treated wounds and CMC control compared to the becaplermin treated wounds (panels d and e vs. panel f). (FIG. 8C) Anti-Calprotectin antibody for macrophage staining (red arrows) shows more inflammatory cells are present in the CMC control compared to either the F-5 treated wounds or becaplermin treated wounds (panels h and I vs. panel g). (FIG. 8D) Picrosirius Red staining with polarized light microscopy confirmed better organized dermis in the F-5-treated wounds than the CMC control or becaplermin treatment (pane k vs. panel j and panel 1). (FIGS. 8E and 8F) Quanitative data for PECAM-1 positive staining per high powered field (HPF) (E) is given as well as the number of macrophages per HPF (F). * p #0.05, ** p #0.01 and *** p #0.001. The above data represent a consensus from multiple and non-continuous sections of a given skin specimen.

FIGS. 9A-9D show a 27-amino acid peptide, F-8, determines the wound healing effect of Hsp90α. (FIG. 9A) A schematic representation of mutagenesis of Hsp90α down to the minimum peptides of 27 amino acids and the profiles of their pro-motility activities. (FIG. 9B) GST-fusion proteins were generated as shown in a SDS gel stained with Coomassie blue. (FIG. 9C) Effects of the various GST-fusion proteins (300 μg/ml for all) on wound healing in pigs, which followed the procedures as shown in FIG. 4. (FIG. 9D) Quantitation of the wound healing data in triplicate. * p #0.05 compared with placebo.

FIGS. 10A-10E show the comparison of F-5 on human versus pig cell migration. Primary human and pig keratinocytes and dermal fibroblasts were isolated. (FIG. 10A) Motility of the serum-starved human and pig keratinocytes on type I collagen in response to FBS (10%), Hsp90α (10 μg/ml) or TGFa (20 ng/ml). (FIG. 10B) Motility of human and pig dermal fibroblasts in response to FBS (10%), Hsp90α (10 μg/ml) or PDGF-BB (15 ng/ml). Quantitative analysis of the above migration assays is presented as Migration Index (%). (FIG. 10C) Lysates of the normal (Nor) and diabetic (Db) human and pig dermal fibroblasts were subjected to anti-LRP-1 antibody immunoblotting. (FIG. 10D) Down-regulation of LRP-1 in both human (lane 3) and pig (lane 6) dermal fibroblasts was confirmed by Western blot with anti-LRP-1 antibody. (FIG. 10E) The cells shown in panel D were subjected to colloidal gold migration assay in response to F-5 or PDGF-BB stimulation. Down-regulation of LRP-1 blocked F-5-stimulated (bars 6 and 15), but not PDGF-BB-stimulated (bars 9 and 18), dermal fibroblast migration. This experiment was repeated four times and reproducible results obtained. * p #0.05.

FIG. 11 shows the 27 amino acids of F-8. In addition to albumin, any other protein drug carriers could be used in combination with the F-8 fragment.

### DETAILED DESCRIPTION

Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

The term “non-immunogenic” refers to inability or attenuated ability of a substance, e.g., a protein, a toxin, or a peptide, to provoke an immune response. In one aspect, the immune response is a humoral or cell-mediated immune response.

The term “carrier protein” refers to a protein that transports, diffuse, or deliver a substance into or out of aa cell. In one aspect, the carrier protein can transport specific substances through intracellular compartment, into the extracellular fluid, or across the cell membrane. In another aspect, the substance is a chemical, an amino acid, a peptide, a protein, a lipid, or any biological or non-biological agent.

The term “recombinant polypeptide” or “recombinant protein” refers to a polypeptide which by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

The term “therapeutic,” as used herein, refers to the full spectrum of treatments for a disease or disorder. A “therapeutic agent” of the disclosure may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of a disease or disorder; or may act to minimize the time required, the occurrence or extent of any discomfort or pain, or physical limitations associated with recuperation from a disease, disorder or physical trauma; or may be used as an adjuvant to other therapies and treatments. In one aspect, the therapeutic agent comprises, alternatively consists essentially of, or yet further consists of a chemotherapeutic, a toxin, a radiotherapeutic, a targeting agent, a radiosensitizing agent, a biological agent, an antisense compound, or any agent that has therapeutic effect.

The term “topical administration,” as used herein, refers to delivery of a topical drug or pharmacologically active agent to the skin or mucosa. Topical administration, in contrast to transdermal administration, provides exclusively or predominantly a local rather than a systemic effect. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream.

The term “tissue regeneration,” as used herein, refers to regrowth, renewal, or restoration of a tissue or portion of a tissue from the remaining tissue or organ. In one aspect, the remaining tissue or organ includes a damaged or missing tissue or organ. In another aspect, the tissue regeneration restores the tissue or organ to its original size or shape. In some aspect, the tissue regeneration does not restore the tissue or organ to its original size or shape.

The terms “wound closure” and “re-epithelialization” are used interchangeably and refer to a process that results in a wound or a portion of a wound becoming covered by a sheet of epithelial cells.

The term “progression or conversion,” as used herein, means a process in which certain superficial partial-thickness burns spontaneously advance into deep partial-thickness or full-thickness wounds. In one embodiment, the progression of a wound into deeper tissue affects the results of burn wound treatment.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

An equivalent or biological equivalent nucleic acid, polynucleotide or oligonucleotide or peptide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, oligonucleotide or peptide.

The term “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest.

“Detectable labels” or “markers” include, but are not limited to radioisotopes, fluorochromes, chemiluminescent compounds, dyes, and proteins, including enzymes. Detectable labels can also be attached to a polynucleotide, polypeptide, antibody or composition described herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), infra.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Additional examples of stringent hybridization conditions include: low stringency of incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like In one embodiment, the mammals include horses, dogs, and cats.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Heat shock protein 90α is a chaperone protein and is commercially available from abcam (ab48801). The amino acid sequence of the human protein is available at UniProtKB-P07900, last accessed on Nov. 30, 2015.

The term “albumin” refers to a family of globular proteins, which includes the serum albumins. Albumins are found in blood plasma and may not be glycosylated. The human serum protein is a 65-70 kDa protein, and the sequence is known in the art (see Accession No. NM_000477) or UnitProt P02868. Human serum albumin (HSA, or HA), is disclosed as SEQ ID NO:1038 in US Patent Publ. No. 2015/0329616, recombinant production of HA (rHA) in microorganisms has been disclosed in EP 330 451 and EP 361 991. Animal homologs are known in the art.

The term “albumin derivative” intents a modified sequence or variant thereof, e.g., Proalbuin lille (see Abdo, et al. (1981) FEBS Letters, Vol. 131 (2):286) and US Patent Publ. No. 2015/0329616 which discloses albumin fusion proteins.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

### MODES FOR CARRYING OUT THE DISCLOSURE

### Recombinant Therapeutic Polypeptides

This disclosure provides a recombinant polypeptide comprising, or alternatively consisting essentially of, or yet further consisting of, an Hsp90α polypeptide sequence (SEQ ID NO.1), an F-8 polypeptide sequence (SEQ ID NO. 2), an F-5 polypeptide sequence (SEQ ID NO. 3), an F-6 polypeptide sequence (SEQ ID NO. 4: EEKEDKEEEKEKEEKESEDKPEIEDVGS DEEEEKKDGDKKKKKKIKEKYIDQEE), or an equivalent thereof, wherein the recombinant polypeptide is operatively linked to a non-immunogenic carrier protein. The recombinant polypeptide comprise as a minimum amino acid sequence the 27-amino acid sequence of F-8 but may include additional amino acids on either or both the amine or carboxy termini of F-8. In one aspect, the additional amino acids include amino acids from the parent protein Hsp90α, e.g., the amino acids that are included with the polypeptide F-5 fragment consisting of 115 amino acid units which is from amino acid number 236 to 350 of Hsp90α, or alternatively and the F-6 fragment having 54 amino acid units from amino acid number 236 to 289 of Hsp90α (sequence EEKEDKEEEKEKEEKESEDKPEIEDVGSDEEEEKKDGD KIKEKYIDQEE) (SEQ ID NO. 4). In one aspect, the recombinant polypeptide specifically excludes the parent wild-type protein Hsp90α or an equivalent thereof.

In one aspect, the equivalent comprises, or alternatively consists essentially of, or yet further consist of, a polypeptide having at least 70% amino acid identity to SEQ ID NOs. 1, 2, 3, or 4 or a polypeptide that hybridizes to a polypeptide encoded by a polynucleotide that hybridizes under conditions of high stringency to a reference polynucleotide encoding a polypeptide comprising SEQ ID NOs. 1, 2, 3 or 4 or the complement of the reference polynucleotide.

In certain aspects, the non-immunogenic carrier protein is selected from the group of albumin, pro-albumin, an albumin variant or derivative, glutathione S-transferase, serum, soybean trypsin inhibitor, thyroglobulin, ovalbumin, polyethylene glycol (PEG), or keyhole limpet hemocyanin. The non-immunogenic carrier protein is selected based on the subject to be treated, e.g., a human albumin would be selected for treating a human patient. In a particular aspect, the non-immunogenic carrier protein comprises, or alternatively consists essentially of, or yet further consists of, albumin or a derivative thereof.

The carrier protein is operatively linked to the amine or carboxy termii of the F-8 polypeptide using linker or a variety of chemical modifications to join polypeptides. Methods for joining polypeptides are described in U.S. Pat. No. 6,475,490 and known in the art.

Alternatively, the recombinant polypeptide is prepared by linking the polynucleotides encoding each portion of the protein into one continuous polynucleotide and expressing the polypeptide as a fusion protein. Thus, recombinant expression systems as described below are further provided herein.

### Polynucleotides and Expression Systems

This disclosure also provides a polynucleotide encoding the recombinant polypeptide as described above. The polynucleotides can be contained with a vector that optionally comprises regulatory sequences operatively linked thereto for the expression and/or replication of the polynucleotides. The appropriate regulatory sequences, e.g., promoters, will vary with the sequence (DNA or RNA) and the use of the polynucleotide. Host cells, e.g., prokaryotic (E. coli or other bacteria), eukaryotic (animal or plant) can comprise the polynucleotides, with or without containment within a vector for expression or replication of the polynucleotides.

The polynucleotides of this invention can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this invention also provides a process for obtaining the recombinant polypeptide of this disclosure by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells as described above and constructed using well known methods. See Sambrook and Russell (2001), supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; DEAE-dextran; electroporation; or microinjection. See Sambrook and Russell (2001), supra for this methodology. The recombinant polypeptides can be isolated from the cell culture by use of purification tags or antibodies to the specific portions of the polypeptide, e.g., the albumin or the F-8 portion.

### Compositions and Therapeutic Uses

This disclosure also provides a composition comprising, or alternatively consisting essentially of, or yet further consisting of, any one or more of the recombinant polypeptide, the polynucleotide or the complement thereof, the vector or the host cell, as described above, and a carrier. The composition can further comprise a therapeutic agent other than the recombinant polypeptide, e.g., PDGF. In one aspect, the carrier is a pharmaceutically acceptable carrier. In another aspect, the composition is formulated for topical administration. Non-limiting examples of such include an aqueous solution, a suspension, a dispersion, a salve, an ointment, a gel, a cream, a lotion, a spray, a lyophilized powder, or a paste.

The compositions can additional contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The pharmaceutical compositions can be administered by any one of the following routes: topically, ocular, oral, systemic (e.g., transdermal, intranasal or by suppository), or parenteral (e.g., intramuscular, intravenous or subcutaneous) administration. In some embodiments, the manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another manner for administering the compositions as described herein is topically.

The compositions can be formulated to a define therapeutic strength, e.g., wherein the concentration of the polypeptide is from about 0.025 μg/ml to about 200 μg/ml, or 0.5 μg/ml to about 150 μg/ml, or about 0.1 μg/ml to about 100 μg/ml, or from about 0.3 μg/ml to about 50 μg/ml.

The recombinant polypeptides and compositions as described herein are useful in methods to promote epidermal tissue regeneration and/or re-epithelialization or prevent cell apoptosis in wounded epithelial tissue, in a subject in need thereof, the method comprising, or alternatively consisting essentially of, or yet further consisting of, administering to a tissue in need thereof an effective amount of the polypeptide or composition as disclosed herein, thereby promoting epidermal tissue regeneration and/or re-epithelialization or preventing cell apoptosis in wounded epithelial tissue. In one aspect the subject is a mammal, e.g. a human patient.

The recombinant polypeptides and compositions as described herein are useful in methods to promote wound healing, or to treat or heal wounds in a subject in need thereof, the method comprising, or alternatively consisting essentially of, or yet further consisting of, administering to the wounded tissue in need thereof an effective amount of the polypeptide or composition as disclosed herein. In one aspect the subject is a mammal, e.g. a human patient.

In one aspect, the compositions are topically applied to the area to be treated.

The compositions can be combined with other wound-healing therapeutic agents, e.g., PDFG and the administration of the recombinant polypeptide and the other therapeutic agent is concurrent or sequential.

The recombinant polypeptide or the compositions can be administered as need and determined by the treating physician, e.g., non-limiting treatment regimens include about every 6 to about every 72 hours or about every 24 to about 48 hours.

### Materials and Methods

### Animals

Female Yorkshire pigs (S&S Farms, Ramona, Calif.) 2 to 3 months in age and weighing 20-25 kgs at arrival were acclimated for at least one week prior to experimental procedures. Six normal pigs were used for non-diabetic control wound studies. An additional six pigs were made diabetic by STZ injection, among which one pig died 5 days after STZ induction of unknown etiology. The other five pigs were maintained in a diabetic state throughout the periods of experiments with fasting blood glucose levels above 200 mg/dl.

### Studies in Non-Diabetic Healthy Pigs

During the initial “wound pattern” studies, 2.0 cm×2.0 cm full-thickness wounds were created in healthy pigs exactly following the procedures as described in detail below.

### Induction of Hyperglycemia by STZ in Pigs

STZ (Enzo Life Science, Farmingdale, N.Y.) was prepared in 0.9% saline (Teknova, Hollister, Calif.), sterilized by filtration through a 0.22 mm filter and administered at 150 mg/kg of body weight after the pig was sedated. The intravenous injections were carried out over 15-20 minutes. Fasting blood glucose levels were tested twice weekly with “Freesytle light” glucose monitor and test strips (Abbott Diabetes Care, Alameda, Calif.). The blood glucose levels were sustained between 250 and 450 mg/dl during the course of the experiments whether it was one month or four months, largely by controlling the daily food intake of the animals. Humulin N insulin (Eli Lilly, Indianapolis, Ind.) also was planned to be given intravenously to the pigs if the glucose levels rose to 600 mg/dl or higher in order to avoid possible ketoacidosis. To test the blood levels of A1c, 1 cc of blood was collected from the ear vein and tested by Antech Diagnostics (Irvine, Calif.) for the averaged concentration of glycated hemoglobin. A1c serves as a marker for the average blood glucose levels for the previous period of three months.

### Creating the Pattern of Multiple Wounds for Control and Treatments

A new pattern of wounds for fair comparative studies is proposed. Wounds on one side of the pig were entirely used for control treatments (sterile carboxymethyl cellulose, CMC) and wounds on the other side of the pig used for treatments of the peptides of interest (see the text for details). All surgical procedures took place under sterile conditions in a designated operating room. Animals were pre-medicated with Ketamine/Xylazine 2.2-4.4 mg/kg. Once sedated, animals were intubated and maintained with 1-4% Isoflurane continuous inhalation. Intramuscular injections of Bupronorphene at 0.02-0.05 mg/kg and Carprofen tablets at 2-4 mg/kg were used as post-operative analgesics. Under anesthesia, the pig's sides were shaved and prepared with betadine scrub and solution. Wounds were created on day 14, 20, 45 or 90 following STZ injection. Depending upon design of a given experiment, nine (in three rows) to twelve (in four rows) 1.5 cm 61.5 cm squares were outlined using permanent black marker around a premade template on the pig's torso. This area was washed with ethanol and prepped with sterile drapes. The distance between two wounds is 2.5 cm.

Using number 15 scalpel blades the wounds were cut to a full thickness depth; the epidermis, dermis and underlying fat were removed to expose the fascia layer below. The depths of the wounds were measured at approximately 5 mm. In one embodiment, the number of wounds on each side is 12, making total 24 wounds for each surgery in a pig.

FDA-approved becaplermin gel (Regranex from Smith and Nephew, Andover, Mass., or recombinant human PDGF-BB) was used as a positive control for the treatment of diabetic wounds. Recombinant full-length or the F-5 fragment of Hsp90α were mixed in 0.3 gm of 15% sterile CMC. These drug or tested Hsp90α proteins were topically applied on wounds in triplicates in a pig. Wounds were then covered with Opsite clear bandages (Smith and Nephew, Hull, UK), overlaid with a cotton gauze cloth taped to cover the entire wound area. Finally, the entire area that included all the wounds on two sides of the pig is wrapped (360°) in elastic bandages, followed by a final wrap in Elastikon (Johnson & Johnson, New Brunswick, N.J.).

### Measurements of Wound Healing

After surgery and various treatments, digital photographs were taken individually of each wound on day 0, 7, 14, and 21 from a fixed distance. Wound healing was analyzed based on measurements of the wound closure and histology/immunohistochemistry. To measure wound closure, the area of an open wound on that day was measured and compared to the area of the wound on day 0 following surgery, using the software AlphaEase FC version 4.1.0 (Alpha Innotech Corporation, Miami, Fla.), as previously described [8, 9]. The histological analyses were carried out for skin wounds and the pancreas. Wedge biopsies measuring 2 cm×2 cm were taken on day 14, 21 or otherwise specified day for skin wounds. The pancreas was removed on the day when the animal was sacrificed. All tissue samples were fixed in 10% formalin (VWR, Randor, Pa.), and placed in paraffin blocks for sectioning. Immunohistochemistry studies on skin wounds were conducted with anti-PECAM-1 (1:100, SC-1506, Santa Cruz Biotechnology, Santa Cruz, Calif.), anti-AGE (1:1000, ab23722, Abcam), anti-pan keratin (1:100, ab8068, Abcam) and anti-calprotectin molecule for macrophage staining (1:100, mA1-80446, Thermo Fisher Scientific, Rockford Ill.) antibodies. Sections of the pancreas from normal and diabetic pigs were immunostained with anti-insulin antibody (1:50, GTX73558, GeneTex), according to previously detailed procedures [8].

Sections were also stained via Picrosirius Red and H&E. PECAM-1 (capillary lumen) density in the wound beds were measured as the average number of PECAM-1 positive lumens from five high powered fields (HPF, 20×) per section. Analysis was performed by a pathologist who was blinded to the treatment groups. Similarly, the numbers of macrophages in seven high powered fields (HPF, 20×) were averaged [38].

Isolation of Primary Skin Cells from Pigs and Humans

Human skin samples from patients obtained with informed consent for elective surgeries were washed with PBS then placed in PBS containing 25 caseinolytic units/ml of dispase (BD Bioscience, San Jose, Calif.) and incubated overnight at 4° C. The skin samples were washed with PBS and the epidermis was separated from the dermis by a set of sterilized surgical tools. To isolate keratinocytes, the epidermis was placed in 0.25% trypsin-EDTA solution (Gibco, Life Technologies, Grand Island, N.Y.) for 20 minutes at 37° C. and the digestion reaction stopped by the addition of soybean trypsin inhibitor. The cell and tissue mixture was poured through a cell strainer and spun down (1300 g, 3 minutes). The cell pellets were re-suspended in and washed with PBS. After a final spin down, keratinocyte media containing 1% gentamycin was used to re-suspend the cells and then plated in tissue culture dishes pre-coated with rat tail type I collagen (29 mg/ml, BD Bioscience). To isolate dermal fibroblasts, the dermis section was minced and placed in collagenase (1000 units/ml, Alfa Aesar, Ward Hill, Mass.) for 2 hours at 37° C. The tissue and cell mixture were passed through a cell strainer. Cells were spun down and washed with PBS. Cell pellets were plated with 20% fetal bovine serum (FBS)-containing DMEM medium. After the majority of cells have attached, the amount of FBS was reduced from 20% to 10%.

The isolation of pig keratinocytes follows the same procedures as above for human keratinocytes except the media is supplemented with a higher concentration of calcium (0.3 mM [Ca2+]) [39]. Without being bound by a theory, pig keratinocytes were more sensitive to the human keratinocyte media and were unable to survive beyond the initial attachment of the cells. By testing varying concentrations of calcium, we found that 0.3 mM [Ca2+] provided the best support for pig keratinocyte growth. Pig dermal fibroblasts were able to survive and expanded for several passages using the same media as human dermal fibroblasts.

### Protein Purification

cDNA cloning, production and purification of recombinant Hsp90α proteins have been carried out as previously described [7, 9].

### Recombinant Polypeptide Production

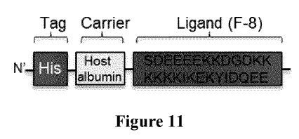

F-8 was linked to pig (NM_001005208, Size: 1902 bp) and human (NM_000477.5, cDNA Size:1830 bp) albumin cDNAs by one of the four mechanisms used for drug development, called “drug-HAS conjugate” (see Kratz, F. (2008) Albumin as a drug carrier: Design of prodrugs, drug conjugates and nanoparticles. J. Control. Release, 132, 171-183). The choice of the pig albumin gene is because the preclinical host is pigs but when the use is for human patients, human albumin is used. The experimental strategy is to use a single step polymerase chain reaction (PCR) to clone the albumin gene together with in framed F-8 sequence to the pET-15b His-tag expression vector. To do so, the 5′ primer of PCR contains a designed restriction enzyme site (from pET15b vector) followed by the 18 nucleotides encoding the first six amino acids of the albumin gene. The 3′ primer of PCR, however, contains 18 nucleotides encoding the last 6 amino acids of the albumin gene at the 5′ end followed by 81 nucleotides that encode the 27 amino acids of the human F-8, followed by a designed restriction enzyme site (from pET15b) at the 3′ end. As a control for specificity, the corresponding 27 amino acids from human Hsp90β, F-8β, is cloned and used as the negative control. Vertebrates have two Hsp90 genes, Hsp90α and Hsp90β. A study shows that only topically applied Hsp90α, but not Hsp90β, protein is capable of promoting wound healing (see Jayaprakash et al. (2015) Hsp90α and Hsp90β together operate a hypoxia and nutrient paucity stress-response mechanism during wound healing. J. Cell Scie. 128:1475-80). Within the two corresponding F-8 regions, Hsp90α and Hsp90β differ in seven amino acids throughout the evolution (see Li et al. (2012) Secreted heat shock protein-90 Hsp90 in wound healing and cancer. Biochim Biophys Acta, 1823, 730-41). Without being bound by a theory, within the seven amino acid variants, two lysine residues in Hsp90α, (they are substituted by alanine and threonine residues in Hsp90β determine the wound-healing activity of Hsp90α. Thus, the purpose of having “albumin-F-8β” fusion protein as a control is to ensure that the observed effects come from F-8α, not from the albumin part. A schematic representation of this design is shown in FIG. 11.

The albumin-F8 constructs in pET15 vector can be transformed into the BL-21 bacterium strain for protein production. Studies show the utilization of pET15b system for Hsp90 protein production and purification by Ni⁺ affinity chromatography (Cheng et al. (2008) Transforming growth factor alpha (TGFalpha)-stimulated secretion of HSP90αalpha: using the receptor LRP-1/CD91 to promote human skin cell migration against a TGFbeta-rich environment during wound healing. Mol Cell Biol 28: 3344-3358; Cheng et al. (2011) A fragment of secreted Hsp90αalpha carries properties that enable it to accelerate effectively both acute and diabetic wound healing in mice. J Clin Invest 121: 4348-4361; Li et al. (2007) Extracellular heat shock protein-90αalpha: linking hypoxia to skin cell motility and wound healing. EMBO J 26: 1221-1233) and those methods were followed here. In one embodiment, additional step(s) of protein purification can be added, for example the step of FPLC (fast protein liquid chromatography) protein purification. An additional method to purify Hsp90α protein, is to use the HiLoad 16/60 Superdex 200 pg column (GE) with 60-65 fractions, 2 ml per fraction). The protein purity of FPLC is determined by the densitometry scanning of the ambumin-F8 protein divided by the densitometry scanning number of the entire lane (18 kDa to 250 kDa) in Coomassie blue-stained SDS gel, as described previously in Sahu et al. (2012) Mol Biol Cell, 23, 602-13. An additional ion-exchange chromatography can be used to achieve the desired purify of certain fragments of Hsp90α protein. An additional cation exchange chromatography (HiTrap SP HP, GE) FPLC step has made the purity of these proteins to >90%. For both in vitro and in vivo experiments, purified proteins will all be adjusted to the final concentration of 1 μg/ml in PBS with 1% glycerol, divided to aliquots and stored at −80° C.

### Cell Migration Assays

The colloidal gold migration assay was conducted as described previously [37]. Data from independent experiments (n>3) were averaged and calculated (mean SD, p, 0.05). In addition to rat tail collagen I, porcine collagen type I/III (45%/45%) from YO Proteins (Huddinge, Sweden) was also used in pig cell migration assays as a comparison.

### Statistic Analyses

Data on animal wound healing were based on three or more independent experiments, multiple diabetic and control pigs. Data are presented as mean +/−standard deviation (s.d.). Statistical significance for comparisons was determined by the Student's two-tailed t-test. A p value equal or less than 0.05 was considered statistically significant [40, 41].

### Ethics Statement

All animal studies were according to a porcine animal protocol approved by the University of Southern California's Institutional Animal Care and Use Committee (Protocol #11581). Early termination, if necessary, of animals was in accordance with USDA's currents space requirements found in the 8th Edition of the Guide for the Care and Use of Laboratory Animals.

Human skin samples from patients were obtained under the protocol HS-11-00156 “Isolation of primary cells from various human tissues.” Samples were obtained during elective surgeries and are de-identified. These samples are to-be-discarded waste tissue from the operating room and therefore no formal consent is given. Patient information is not collected nor recorded. The protocol and consent procedures were approved by the University of Southern California Health Sciences Campus Institutional Review Board.

### Results

Comparisons of Skin from Four Preclinical Models

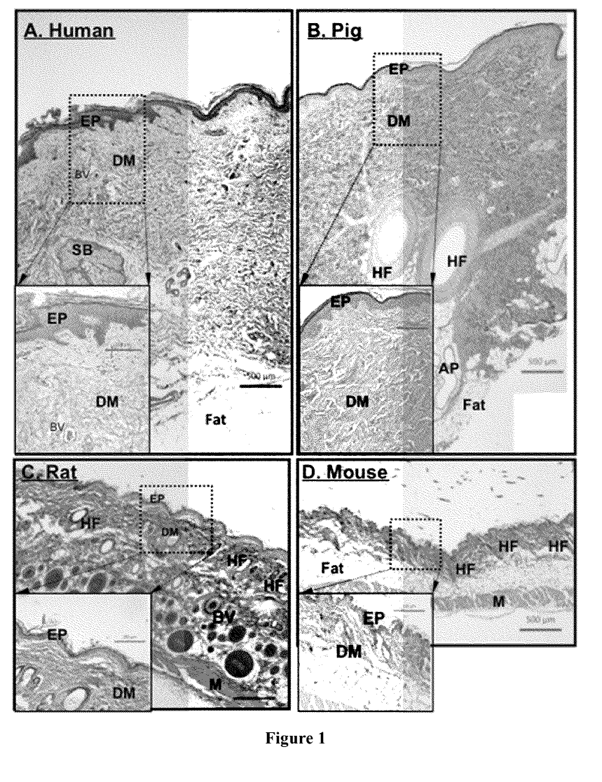

Simultaneously, the histology of vertical sections of skin from humans, pigs, rats and mice was evaluated. The biopsies from rodents and pigs are from similar sites along the spinal region, while the human samples came from different locations due to the variability of the surgical procedures. Nonetheless, similar results were obtained as those presented in FIG. 1. Human and pig skin share many similarities in their overall thickness and architecture, including a clear division between the epidermis and the dermis and appearance of appendage (hair follicles, sweat glands, sebaceous glands) distribution (panels A vs. B).

Compared to humans, pigs have less vasculature in their skin and pigs do not have eccrine sweat glands [10] whereas humans have both eccrine and aporcine sweat glands. Rodents have eccrine glands in their foot pads only [11]. The total epidermis of pigs measures 30-140 mm, while human epidermis measures 50-120 mm, rodent epidermis on the other hand measures only 10-45 mm [1, 12, 13]. The turnover time of the epidermal layer is approximately 30 days for pigs, 26-28 days for humans [14, 15] but only 8-10 days for rodents [16, 17]. The combined thickness of the epidermis and dermis in rodent skin is about 10% to 15% that of human skin (FIG. 1, panels C and D vs. A and B). The outer most layer of the epidermis is the stratum corneum, here the number of cell layers is similar between pigs and humans; pigs having anywhere between 10-25 layers depending on anatomical location [18], while humans average around 15-25 cell layers [19]. Rodents though typically have only 5 cell layers [20] in their stratum corneum. The turnover rate of the stratum corneum cells is 16 days for pigs [15] and 17 days for humans [21].

Not only is the skin architecture similar between pigs and humans, but so is their wound healing processes. Wound contraction accounts for 90% of wound healing in rodents, while it only accounts for 50% in pigs and 25-50% in humans [22]. The wound contraction in rodents is due to the presence of the subcutaneous panniculus carnosus layer, which is not found in pigs or humans[1]. It is therefore the similarities between the architecture and wound healing processes between humans and pigs that make them an ideal model.

### Relationship Between Locations and Healing Rates of Wounds in Pigs

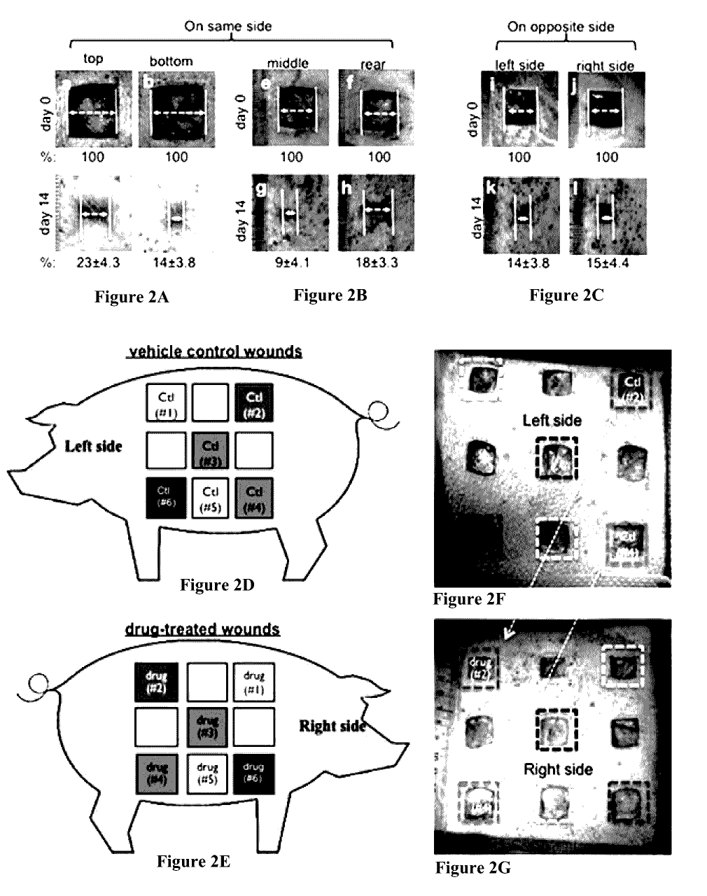

There has been variability in the utilization of pigs as a wound healing model in different laboratories and there is a need to establish a standard procedure to create and treat pig skin wounds. Specifically, along the left or right side of the torso where the wounds are created, the elasticity, thickness and hair follicle density differ from both top to bottom and from left to right, even within the distance of a few centimeters. The epidermis of the skin becomes more pliable going from the top to the bottom side of the torso. As shown in FIG. 2A, wounds that are only a few centimeters from top to bottom (panels a and b) showed significantly different healing rates (panel c vs. panel d). Similarly, as shown in FIG. 2B, wounds that are a few centimeters apart from left to right (panels e and f) on the same side of the torso showed different healing rates (panel h vs. panel g). In contrast, wounds created at similar locations, but on the opposite side of the torso, underwent healing at a similar rate (FIG. 2C, panel k vs. panel l). Quantitation of the representative experiment is shown as a percentage of the unhealed wound (%) below images. In addition, due to the constant movement of the animal (standing and lying down, running around and scratching against enclosure), exchanges by diffusion could occur between drug-treated and placebo-treated wounds on the same side of the torso. As schematically shown in FIGS. 2D and 2E, wounds should be created on opposite sides of the torso and the wounds should not be randomly matched for a treatment versus its control. Instead, as indicated by the same colored boxes, matching wounds for treatment and control should be at corresponding but opposite sides of the pig's body. FIGS. 2F and 2G show that real wounds were created on both sides of a pig, in which two matching wounds are marked with the same colored squares for a given treatment and its control.

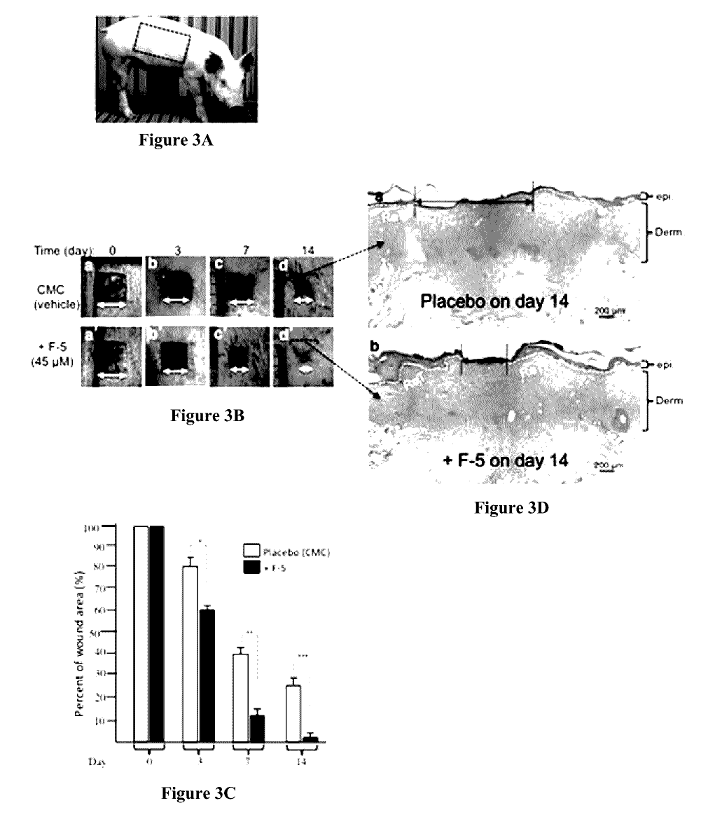

To verify the above design, the wounds were topically treated with recombinant F-5 (amino acids 236 to 350) of human heat shock protein-90αalpha (Hsp90α), which has been previously shown to accelerate wound closure in mice [8, 9]. As shown in FIG. 3B, topical application of F-5 on day 0 accelerated the wound closure on day 3, 7 and 14 (lower panels, a′ to d′), compared to the control (CMC vehicle) treatment (upper panels, a to d). Quantitation of the data is shown in FIG. 3C, which clearly indicates the wound healing-promoting effect of F-5.

More encouragingly, H&E staining of the wounds on day 14 revealed that F-5 accelerated the re-epithelialization process, i.e. lateral migration by the keratinocytes at the wound edge, compared to the control (FIG. 3D, panel b vs. panel a).

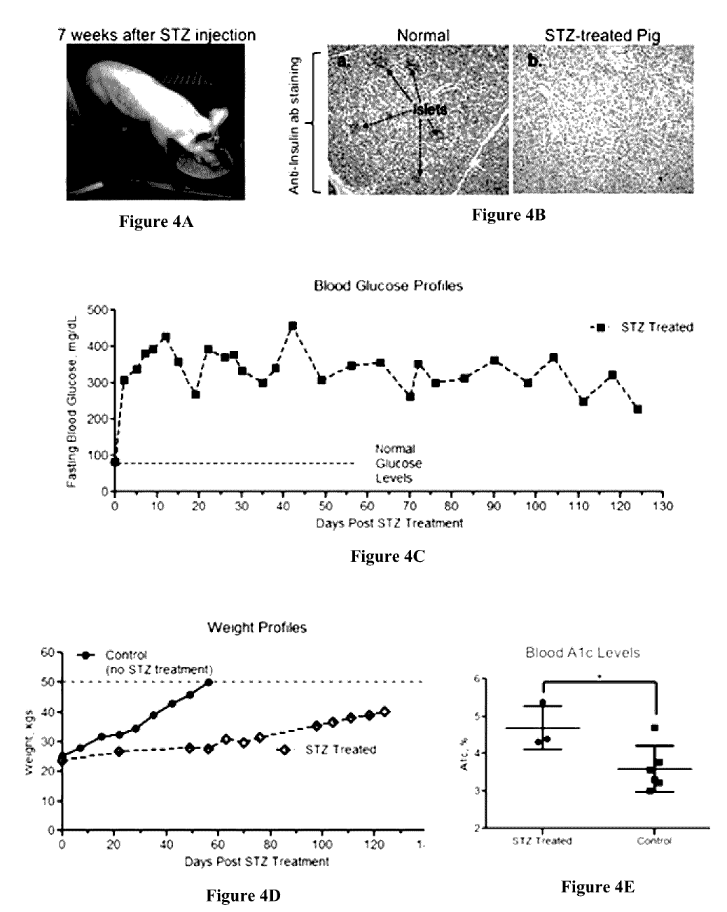

Shared parameters of human diabetes by STZ-treated pigs Streptozotocin (STZ) enters beta (β) cells through the glucose transporter 2, GLUT2, and causes beta cells to undergo destruction via necrosis, resulting in diabetes in many animal species [23]. Nevertheless, a comprehensive analysis of STZ-induced diabetes in pigs with the defined parameters in diabetic humans was not available in wound healing studies. The following were examined: insulin-producing islets in the pancreas, blood glucose profiles, body weight profiles and blood A1c (hemoglobin A1c) levels in pigs following STZ injection up to four months. As shown in FIGS. 4A-4E, a representative farm pig appeared normal 7 weeks after intravenous injection with STZ and its completion of the first wound healing study between week 2 and week 4 (see healed wound marks on the right side of its torso). Sections of pancreases removed from either normal or STZ-treated pigs were subjected to anti-insulin antibody immunostaining analysis, which showed complete destruction of the insulin-producing islets from the STZ-treated pig in comparison to a control pig (FIG. 4B, panel b vs. panel a). The blood glucose level rose rapidly to 300 mg/dl within 24 hours following STZ injection and maintained hyperglycemic levels up to four months, whereas normal pigs kept normoglycemia as expected (FIG. 4C, red dotted line).

Normal pigs that reached the 50 kg weight limit within 60 days were euthanized in accordance with USDA's current space requirements found in the 8th Edition of the Guide for the Care and Use of Laboratory Animals. STZ-treated pigs gained weight slower than non-diabetic pigs, resembling human diabetic patients (FIG. 4D). The blood A1c level of the STZ-treated pigs increased to an average of 4.6%, in comparison to an average of 3.6% in non-diabetic pigs (FIG. 4E).

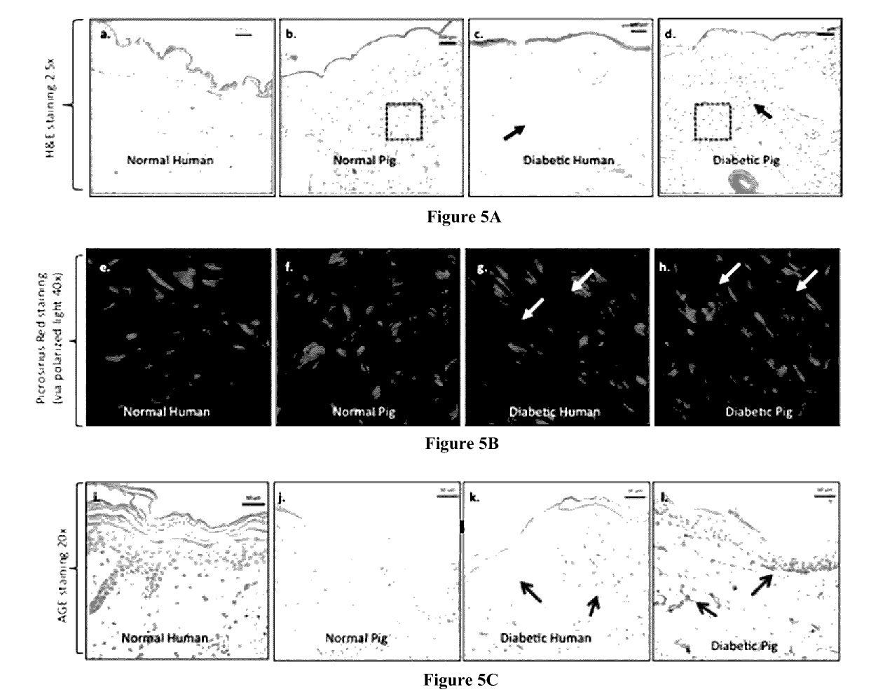

Furthermore, H&E histology, Picrosirius Red staining and AGE immunohis-tochemistry staining analyses showed that diabetic pig skin underwent changes similar to those in diabetic human skin. As shown in FIG. 5A, H&E-stained diabetic human skin showed less density in the dermal connective tissue (black arrow, panel c) than normal human skin (panel a). The pixel density of “white space” was measured in 3 equally sized fields of the dermis for each of the different skin samples (no skin appendages were present in the fields measured, see boxes in FIG. 5, panels b and d). Density measurements for the normal human sample are 672+/−37.7 white pixels (wp)/field (f) versus 1450+/−124.9 wp/f in the diabetic human sample. Similarly, STZ-treated pig skin also exhibited less density of the dermal connective tissues (black arrows) than normal pigs (panel d vs. panel b) although not as strikingly as with the human samples, most likely due to the shorter period of time the pig had been in a hyperglycemic state. The white space density for the normal pig is 639+/−51.6 wp/f versus 818+/−38.1 wp/f. These observations were further confirmed by staining collagens with Picrosirius Red and visualized under polarized light. As shown in FIG. 5B, the collagen in the dermis of diabetic human (panel g) and diabetic pig (panel h) were disorganized, in comparison with the non-diabetic skin controls (Panels e and f). Moreover, both diabetic human and diabetic pig skin showed increased glycation levels. As shown in FIG. 5C, AGE staining showed overall increased glycation in diabetic human skin (panel k vs. panel i). Similarly, in diabetic pig skin, a scattered but significant increase in glycation, again likely due to the much shorter period of hyperglycemia, was clearly detected (panel l), in comparison to the control (panel j). These results indicated that STZ-treated pigs exhibited the characteristics of diabetes similar to those in humans.

Unexpectedly, Applicant discovered that treatment and control wounds should be on the opposite and corresponding sides of a pig and demonstrates a strong correlation between duration of diabetic conditions and the length of delay in wound closure. Using these new models, the minimum therapeutic entity of secreted Hsp90α is identified to a 27-amino acid peptide, called fragment-8 (F-8). In addition, results of histochemistry and immunohistochemistry analyses reveal more organized epidermis and dermis in Hsp90α-healed wounds than the control. Finally, Hsp90α uses a similar signaling mechanism to promote migration of isolated pig and human keratinocytes and dermal fibroblasts. The result shows standardized pig models for acute and diabetic wound healing studies are useful for testing both an approved drug and an unproved therapeutic agent.

### Identification of the Onset for Delayed Wound Healing in STZ-Treated Pigs

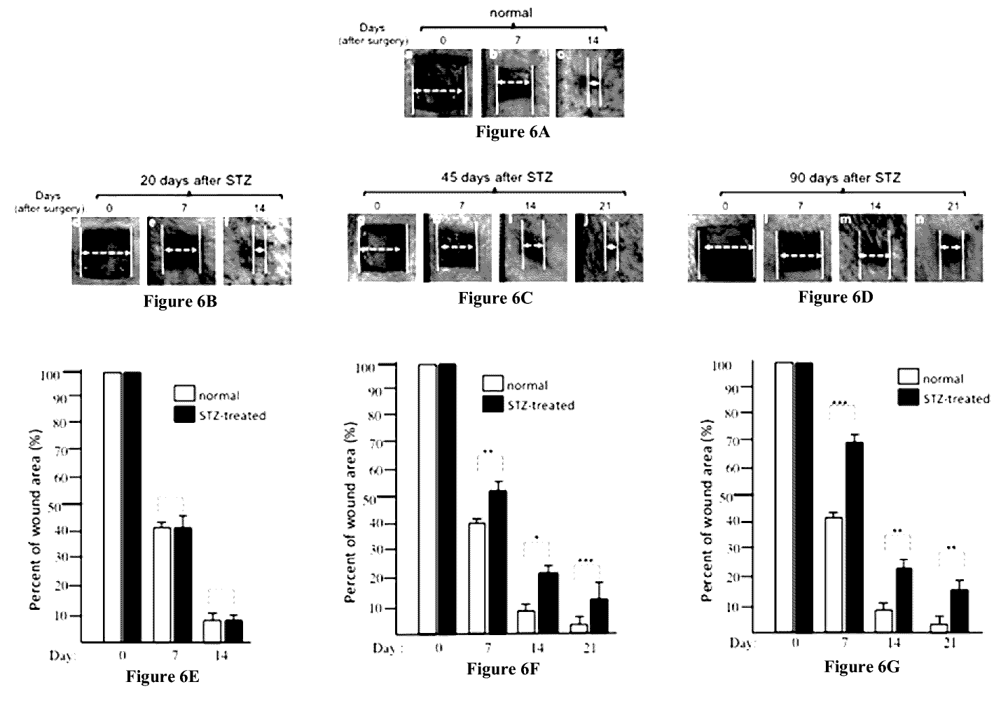

Delayed healing is the clinical signature of diabetic skin wounds in humans. A previous study reported 4 to 6 days of delay in skin wound closure in pigs that were wounded 14-20 days following STZ injection [24]. Without being bound by a theory, it might need to take a longer period of time for diabetic conditions to cause any significant delay in wound healing. To test this hypothesis, a series of wound healing experiments were conducted in pigs whose diabetic conditions were kept for 20, 45 and 90 days prior to the surgical procedures. As shown in FIG. 6A, 1.5 cm×1.5 cm full thickness wounds in control pigs healed around day 14 (panels a, b, c). Similar wounds did not show any significant delay in the pigs 20 days following STZ injection (FIG. 6B, panels d, e, f), after quantitation (6E). However, a significant delay in wound closure was detected in pigs 45 days following STZ injection (FIG. 6C, panels g, h, i, j vs. panels a, b, c). The wounds clearly remained open on day 14 (panel i) and closed around day 21 (panel j). Quantitation of the data showed 12-15% delay in wound closure (FIG. 6F). More convincingly, in pigs 90 days following STZ injection prior to surgery, the wounds remained open even on day 21 (FIG. 6D, panel n). Quantitation showed 18-30% delay in wound healing (FIG. 6G). The experiments could not continue beyond 90 days following STZ injection, due to the 50 kg weight limit set by the USDA space requirements for pigs.

### F-5 Versus Becaplermin (PDGF-BB) Gel on Promoting Wound Healing in Diabetic Pigs

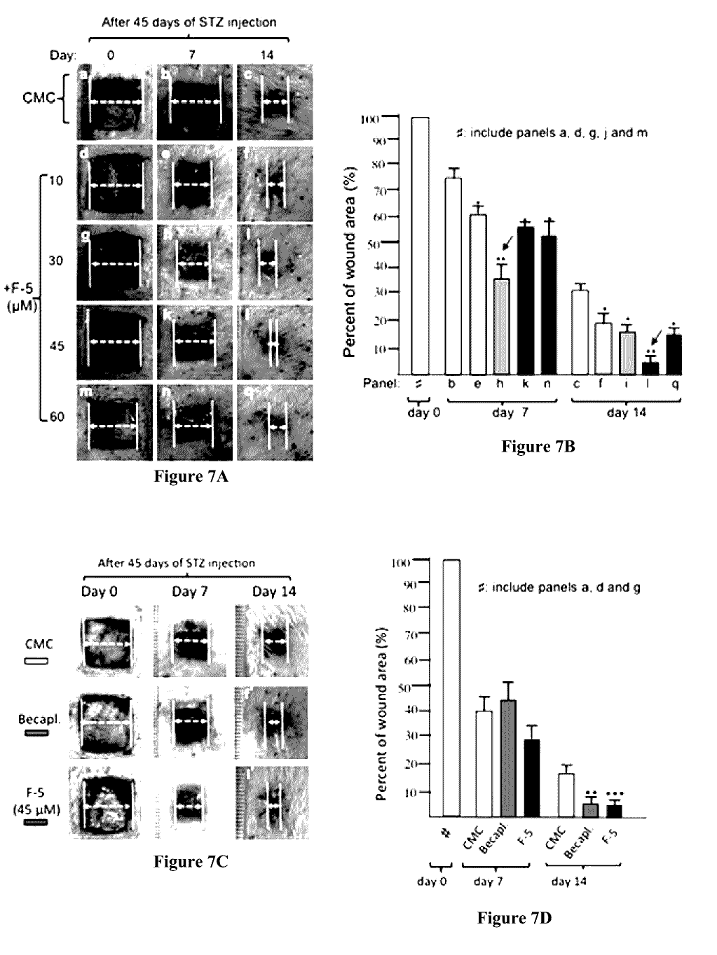

Using the above diabetic pig model, F-5 was also tested for its ability to correct delayed wound healing. On wounds in pigs 45 days following STZ administration, as shown in FIG. 7A, 10 mM of F-5 showed a modest promotion of wound closure on both day 7 and day 14, in comparison to placebo controls (panels d, e, f vs. panels a, b, c). A greater enhancement was observed with 30 mM of F-5, especially on day 7 (panels h vs. panels b and e). The 45 mM F-5 showed rather a weaker stimulatory effect on day 7, with the strongest effect on day 14, leading to complete closure of the wounds (panel l vs. panels c, f or i). The 60 μM F-5 showed little further improvement on day 7 and a slightly inhibitory effect on day 14, in comparison to 45 μM of F-5 (panels n, q vs. panels k, l). Quantitative analyses of these experiments are shown in FIG. 7B. These results indicated a plateau effect for F-5 in diabetic pig wounds between 30 μM to 45 μM.

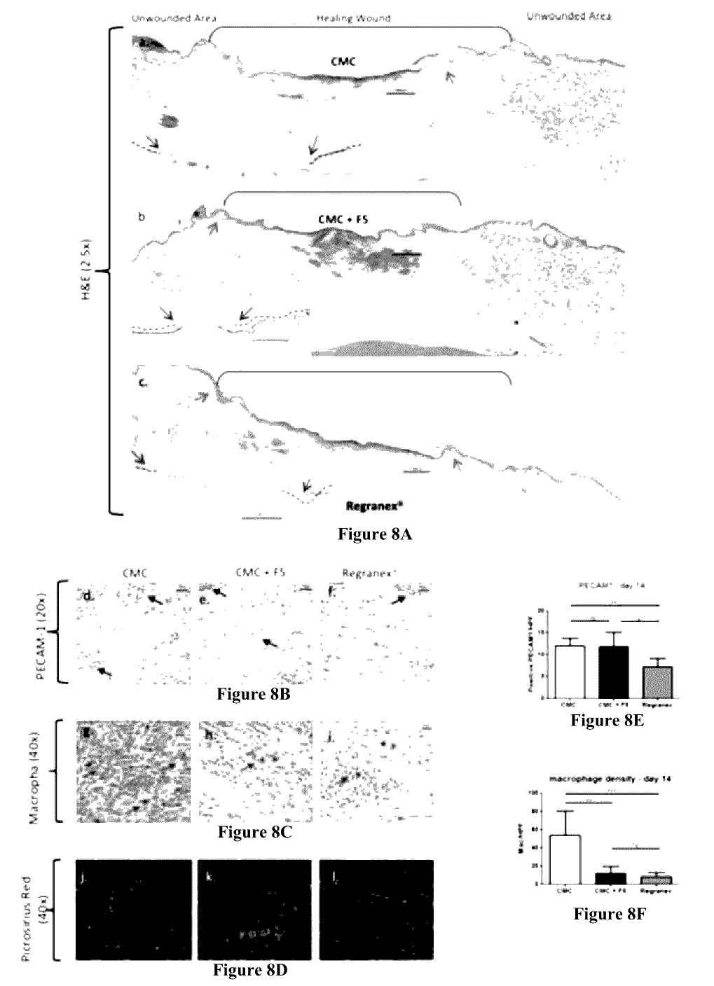

The treatment results of 45 mM dosage of F-5 were compared to the becaplermin gel treatment. As shown in FIG. 7C, delayed wound healing was observed in CMC alone-treated wounds that remained open on day 14 (panels a′, b′, c′). Treatment with becaplermin gel accelerated the wound closure on day 7 and day 14 (panels d′, e′, f). In parallel, treatment with F-5 showed a comparable stimulatory effect on day 7 to becaplermin gel (panel h′ vs. panel e′) and a stronger effect on day 14 than becaplermin gel (panel l′ vs. panel f). Quantitative analysis of these results is shown in FIG. 7D. Interestingly, as shown in FIGS. 8A-8F, histochemistry and immunohistochemistry analyses showed that the F-5-treated wounds exhibited enhanced re-epithelialization (insert FIG. 8A, panel b), more organized collagen deposition (FIG. 8B, panel k) than becaplermin gel or the CMC alone treated wounds. Also, both F-5 and CMC control treated wounds had a similar amount of blood vessel formation over becaplermin treated wounds (FIG. 8B, panels d and e vs. f). Finally, both F-5 and becaplermin treated wounds showed less inflammation with fewer macrophages present in the wound bed over the CMC alone treated wounds (FIG. 8C, panels h and i vs. g).

### 27-Amino Acid Peptide, F-8, Determines Extracellular Hsp90α Function in Wound Healing

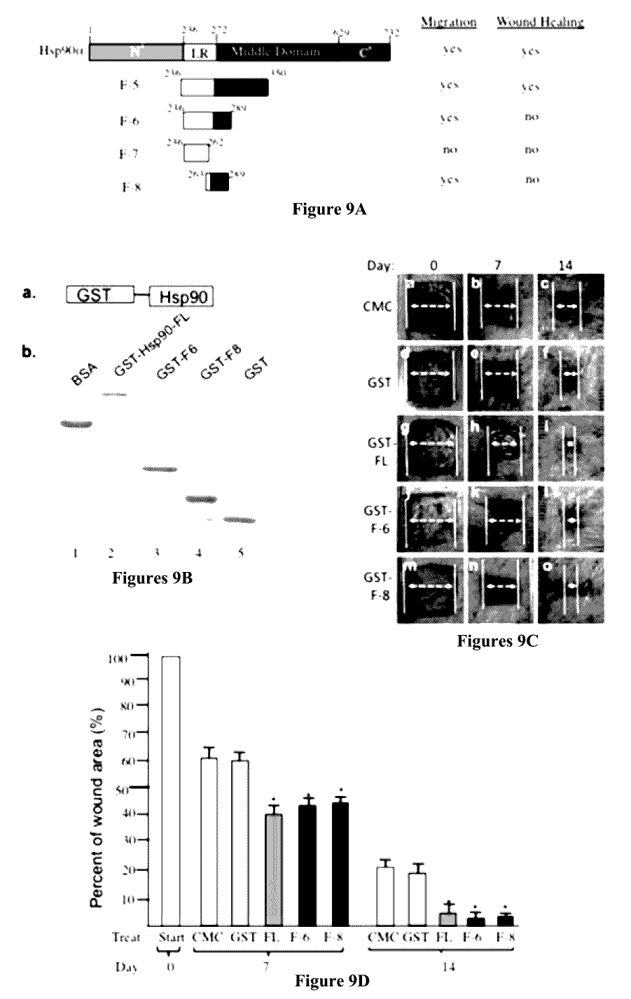

A peptide that reaches its minimum size and still retains its function is preferred for therapeutic development, because of its higher specificity and lower off-target effects, especially when an unrelated carrier protein can be used to correct its possibly compromised stability. This concept prompted us to further identify the minimum size of Hsp90α that still retains the pro-motility activity of Hsp90α in vitro and enhanced wound healing effect in vivo. Deletion mutagenesis of the F-5 fragment, as schematically summarized in FIG. 9A, led to the 54-amino acid peptide, called F-6, that retained the pro-motility effect of F-5. When F-6 was further shortened into two 27-amino acid peptides, called F-7 and F-8, F-8, but not F-7, still retained a majority of the pro-motility activity of F-5. The results also shows that neither F-6 nor F-8 peptide alone was able to promote pig wound healing.

To test the possible compromised stability of these peptides in the wound environment, glutathione s-transferase (GST) was linked to F-6 and F-8 as a carrier protein. As shown in FIG. 9B, GST-FL (Hsp90α), GST-F-6, GST-F-8 and GST alone were shown in an SDS-PAGE gel with BSA as control. Interestingly, when GST-F-6 and GST-F-8 proteins were topically applied to normal pig wounds, as shown in FIG. 9C, both GST-F-6 (panels j, k, l) and GST-F-8 (panels m, n, o) were able to promote wound healing as much as GST-FL Hsp90α did (panels g, h, i), in comparison to the CMC control (panels a, b, c) or GST alone (panels d, e, f). Quantitation of the data is shown in FIG. 9D.

### Hsp90α Promotes Pig and Human Cell Migration Via LRP-1 Receptor

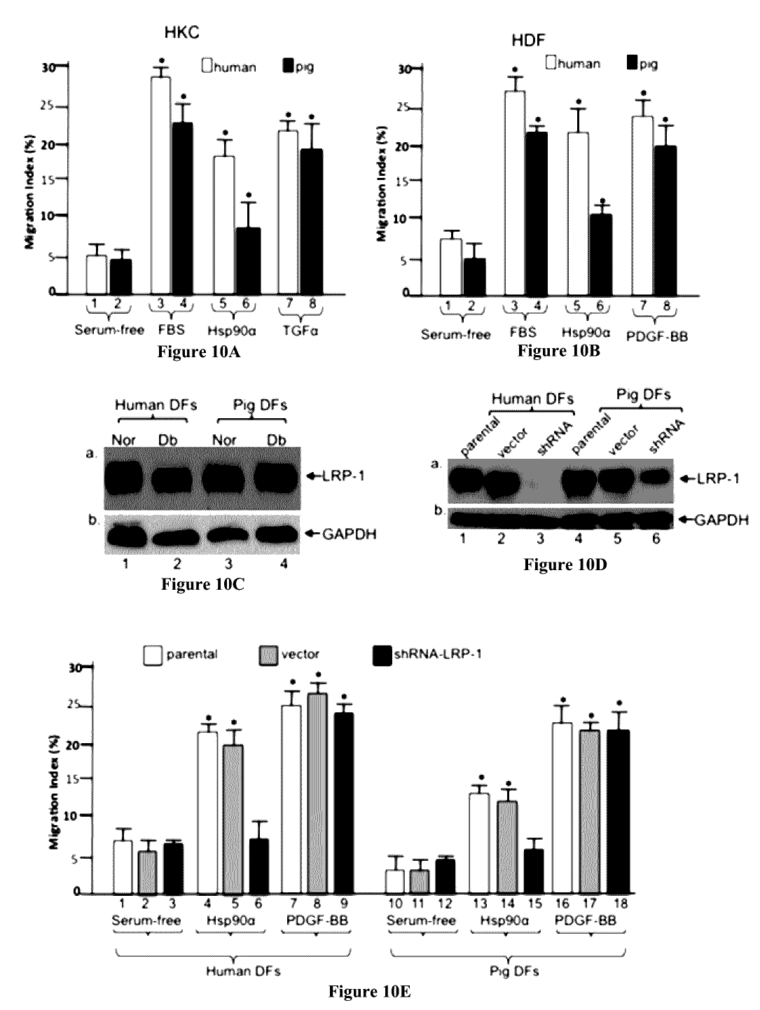

Hsp90α was tested for its ability to promote pig cell migration like it does to human cells in vitro and, more importantly if it uses a similar mechanism. Pig and human keratinocytes and dermal fibroblasts were isolated from surgical specimens and subjected to migration assays. As shown in FIG. 10A, FBS and TGFa equally stimulated both pig and human keratinocyte migration (bars 3, 4 and 7, 8 vs. bars 1 and 2). Interestingly, Hsp90α-stimulated human keratinocyte migration appeared to be significantly stronger than Hsp90α-stimulated pig keratinocyte migration (bar 6 vs. bar 5). Similar observations were made for dermal fibroblast migration. As shown in FIG. 10B, while FBS and PDGF-BB stimulated pig and human dermal fibroblast migration to a similar degree (bars 3, 4 and 7, 8 vs. bars 1 and 2), Hsp90α stimulated a much stronger migration effect of human dermal fibroblasts than pig dermal fibroblasts (bar 6 vs. bar 5). If one extrapolates these results, in conjunction with the F-5 and PDGF-BB stimulated wound healing (see FIG. 7C), it suggests that F-5 may only have shown half of its real efficacy in the pig wounds than it might be able to show in human wounds.

Finally, Hsp90α was also studied for its ability to stimulate pig and human cell migration via the same mechanism, i.e., LRP-1 (LDL-receptor-related protein-1). As shown in FIG. 10C, human and pig dermal fibroblasts (normal or diabetic) express similar levels of LRP-1 (panel a), the cell surface receptor for Hsp90α [7]. Using lentiviral infection, as shown in FIG. 10D, nearly complete knockdown of LRP-1 expression was achieved in human cells (lane 3) and approximately 70% knockdown of LRP-1 expression in pig cells (lanes 6), in comparison to their corresponding human (lanes 1, 2) and pig (lanes 4, 5) controls. When these cells were subjected to the colloidal gold migration assay, as shown in FIG. 10E, Hsp90α was no longer able to promote migration of the LRP-1-downregulated human (bar 6 vs. bars 4 and 5) and pig (bar 15 vs. bars 13and 14) cells. In contrast, down-regulation of LRP-1 did not affect PDGF-BB-stimulated human (bar 9 vs. bars 7 and 8) and pig (bar 18 vs. bars 16 and 17) cell migration. These results clearly indicated that Hsp90α uses a similar signaling mechanism to promote pig and human cell migration.

### Discussion

Wound healing studies in live animals remain the most predictive method to gain insights into the wound healing mechanisms in humans. Among the currently used animal models, it is widely accepted that pigs have skin that is both histologically and functionally closer to humans' and, therefore, have widely been regarded as the “right” model for wound healing studies [1]. Since 2008, for instance, there have been a handful of wound healing studies using STZ-induced diabetic pigs and yet the reported findings were inconsistent [24-30]. Studies from Eriksson's group created full-thickness skin wounds in diabetic pigs 14 days after STZ injection. Based on Hematoxylin and Eosin (H&E) staining, they reported that complete re-epithelialization of 1.5 cm 61.5 cm full thickness wounds occurred on day 12 to 14 for non-diabetic pigs and on day 18 for STZ-treated pigs [24]. Bergmann et al compared partial-thickness wounds on normal and diabetic pigs and reported little difference in wound closure rates [28]. Except for these two studies, the majority of other studies only examined STZ-induced diabetic pigs and did not make any comparisons to normal healthy pigs or did not pay particular attention to whether or not wound healing was delayed in those pigs [25-27]. Consequently, the results of initial experiments that followed the procedures in those studies were highly variable and un-reproducible. Without being bound by a theory, there was a need to first establish the methodology of using pigs as a wound healing model. The current study systematically evaluated all critical parameters and standardized both healthy and diabetic wound healing models in pigs. the new standards include (i) a wound-creating pattern for therapeutic treatment versus the control; (ii) measurements of the physiological parameters of diabetes, (iii) demonstration of effectiveness for a FDA-approved wound healing agent to support the relevance of these models; and (iv) most importantly, identification of a 27-amino acid peptide, called F-8, as the core entity of Hsp90α to promote wound healing.

What is the physiological relevance of Hsp90α secretion to diabetic wound healing? The answer points to the levels of the key cellular responding protein to environmental hypoxia, the hypoxia-inducible factor-1alpha (HIF-1α). Hypoxia-driven secretion of Hsp90α is under direct control of cellular HIF-1α levels [9, 31]. Impaired reaction to hypoxia is known to contribute to impaired wound healing, such as in diabetic ulcers [32]. Lower levels of HIF-1α protein were found in foot ulcer biopsies in patients with diabetes, in which hyperglycemia was shown to reduce the HIF-1α stability and function [33-35]. These findings suggest that delayed diabetic wound healing is the result of HIF-1α destabilization and provide strong support for topical treatment of diabetic wounds with recombinant Hsp90α protein to bypass the damaged HIF-1α in human diabetic wounds. While it remains to be tested whether the HIF-1α levels are affected in this diabetic pig model, this disclosure clearly shows that the topical application of Hsp90α proteins greatly accelerated wound closure in these pigs.

It was argued that the available diabetic animal wound healing models only demonstrate a short-term impairment in the wound repair process and, therefore, may not reflect the true nature of chronic wounds in humans that can persist for years. Hence these diabetic wound models are actually models for impaired acute wound healing rather than true chronic wounds [36]. Given the life span of current experimental animals (i.e., only a fraction of humans') and the variability in the biology of human wounds, it is true that there is no perfect animal model for human skin wound healing studies. The results herein show that the longer the condition of diabetes is sustained in pigs the more evident a delay in wound healing takes place. This finding is consistent with the clinical observations on diabetic foot ulcers in humans.

Therefore, the disclosure provides a method to promote epidermal tissue regeneration and/or re-epithelialization or prevent cell apoptosis in wounded epithelial tissue, in a subject in need thereof comprising administering to a tissue in need thereof an effective amount of the recombinant polypeptide. In another aspect, the disclosure provides a method to facilitate healing or treat a wound or injury, comprising administering to a wounded or injured tissue in a subject in need thereof an effective amount of the recombinant polypeptide. In one embodiment, the method further comprises administering an effective amount of a wound-healing therapeutic agent other than the recombinant polypeptide. In another embodiment, the administration of the recombinant polypeptide and the therapeutic agent is concurrent or sequential. In one embodiment, the subject is a mammal.

In some embodiment, the wound is a skin wound or a wound to an eye. In one embodiment, the skin wound is an acute wound, a diabetic wound, or a burn wound. In some embodiments, the acute wound comprises a traumatic wound or a surgical wound. In another embodiment, the method of treating a wound further comprises treating or preventing progression or conversion of the burn wound.

In one aspect, the injury is an eye disease or an eye injury. In one embodiment, the eye injury is a corneal injury or a conjunctival injury. In another embodiment, the eye injury is caused by a penetrating object, a foreign body, a chemical, or burn.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains. Several aspects of the invention are listed below.

Sequence Listing

SEQ ID NO. 1:
MPEETQTQDQPMEEEEVETFAFQAEIAQLMSLIINTFYSNKEIFLRELIS
NSSDALDKIRYESLTDPSKLDSGKELHINLIPNKQDRTLTIVDTGIGMTK
ADLINNLGTIAKSGTKAFMEALQAGADISMIGQFGVGFYSAYLVAEKVTV
ITKHNDDEQYAWESSAGGSFTVRTDTGEPMGRGTKVILEILKEDQTEYLE
ERRIKEIVKKHSQFIGYPITLFVEKERDKEVSDDEAEEKEDKEEEKEKEE
KESEDKPEIEDVGSDEEEEKKDGDKKKKKKIKEKYIDQEELNKTKPIWTR
NPDDITNEEYGEFYKSLTNDWEDHLAVKHFSVEGQLEFRALLFVPRRAPF
DLFENRKKKNNIKLYVRRVFIMDNCEELIPEYLNFIRGVVDSEDLPLNIS
REMLQQSKILKVIRKNLVKKCLELFTELAEDKENYKKFYEQFSKNIKLGI
BEDSQNRKKLSELLRYYTSASGDEMVSLKDYCTRMKENQKEITYYITGET
KDQVANSAFVERLRKHGLEVIYMIEPIDEYCVQQLKEFEGKTLVSVTKEG
LELPEDEEEKKKQEEKKTKFENLCKIMKDILEKKVEKVVVSNRLVTSPCC
IVTSTYGWTANMERIMKAQALRDNSTMGYMAAKKHLEINPDHSIIETLRQ
KAEADKNDKSVKDLVILLYETALLSSGFSLEDPQTHANRIYRMIKLGLGI
DEDDPTADDTSAAVTEEMPPLEGDDDTSRMEEVD
SEQ ID NO. 2:
SDEEEEKKDGDKKKKKKIKEKYIDQEE
SEQ ID NO. 3:
EEKEDKEEEKEKEEKESEDKPEIEDVGSDEEEEKKDGDKKKKKKIKEKYI
DQEELNKTKPIWTRNPDDITNEEYGEFYKSLTNDWEDHLAVKHESVEGQL
EFRALLFVPRRAPFD
SEQ ID NO. 4:
EEKEDKEEEKEKEEKESEDKPEIEDVGSDEEEEKKDGDKIKEKYIDQEE

### REFERENCES

1. Sullivan T P, Eaglstein W H, Davis S C, Mertz P (2001) The pig as a model for human wound healing. Wound Repair Regen 9: 66-76.
2. Brahmatewari J, Serafini A, Serralta V, Mertz P M, Eaglstein W H (2000) The effects of topical transforming growth factor-beta2 and anti-transforming growth factor-beta2,3 on scarring in pigs. J Cutan Med Surg 4: 126-31.
3. Davis S C, Cazzaniga A L, Eaglstein W H, Mertz P M (2005) Over-the-counter topical antimicrobials: effective treatments? Arch Dermatol Res 297: 190-195.
4. Lindblad W J (2008) Considerations for selecting the correct animal model for dermal wound-healing studies. J Biomater Sci Polym Ed 19:1087-1096.
5. Singer A J, Clark R A (1999) Cutaneous wound healing. N Engl J Med 341: 738-746.
6. Bandyopadhyay B, Fan J, Guan S, Li Y, Chen M, et al. (2006) A “traffic control” role for TGFbeta3: orchestrating dermal and epidermal cell motility during wound healing. J Cell Biol 172: 1093-1105.
7. Cheng C F, Fan J, Fedesco M, Guan S, Li Y, et al. (2008) Transforming growth factor alpha (TGFalpha)-stimulated secretion of HSP90αalpha: using the receptor LRP-1/CD91 to promote human skin cell migration against a TGFbeta-rich environment during wound healing. Mol Cell Biol 28: 3344-3358.
8. Cheng C F, Sahu D, Tsen F, Zhao Z, Fan J, et al. (2011) A fragment of secreted Hsp90αalpha carries properties that enable it to accelerate effectively both acute and diabetic wound healing in mice. J Clin Invest 121: 4348-4361.
9. Li W, Li Y, Guan S, Fan J, Cheng C F, et al. (2007) Extracellular heat shock protein-90αalpha: linking hypoxia to skin cell motility and wound healing. EMBO J 26: 1221-1233.
10. Vardixis N J, Brans T A, Boon M E, Marres L M (1997)) Confocal laser scanning microscopy of porcine skin: implications for human wound healing studies. J Anat 190: 601-611.
11. Quick D C, Kennedy W R, Yoon K S (1984) Ultrastructure of the secretory epithelium, nerve fibers, and capillaries in the mouse sweat gland. Anat Rec 208: 491-499.
12. Meyer W, Scharwtz R, Neurand K (1978) The skin of domestic mammals as a model for the human skin, with special reference to the domestic pig. Curr Probl Dermatol 7: 39-52.
13. Monteiro-Riviere N A, Bristol D G, Manning T O, Rogers R A, Riviere J E (1990) Interspecies and interregional analysis of the comparative histologic thickness and laser Doppler blood flow measurements at fice cutaneous sites in nine species. J Invest Dermatol 95: 582-586.
14. Rothberg S, Crounse R G, Lee J L (1961) Glycine-C14 incorporation into the proteins of nermal stratum corneum and the abnormal stratum corneum of psoriasis. J Invest Dermatol 37: 497-505.
15. Weinstein G D (1965) Autoradiographic studies of turnover time and protein synthesis in pig epidermis. J Invest Dermatol 44: 413-419.
16. Potton C S (1975) Epidermal cell production rates. J Invest Dermatol 65: 488-500. Koster M I (2009) Making an epidermis. Ann N Y Acad Sci 1170: 7-10
17. Gray G M, White R J, Williams R H, Yardely H J (1982) Lipid composition of the superficial stratum corneum cells of pig epidermis. Br J Dermatol 106: 59-63.
18. Holbrook K A, Odland G F (1974) Regional differences in the thickness (cell layers) of the human stratum corneum: an ultrastructual analysis. J Invest Dermatol 62: 415-422.
19. Potton C S, Saffhill R, Maibach H I (1987) Measurement of the transit time for cells through the epidermis and stratum corneum of the mouse and guinea-pig. Cell Tissue Kinet 20: 461-472.
20. Finlay A Y, Marshall R J, Marks R (1982) A fluorescence photographic photometric technique to asses stratum corneum turnover rate and barrier function in vivo. Br J Dermatol 107: 35-42.
21. Hayward P G, Robson M C (1991) Animal models of wound contraction. Prog Clin Biol Res 365: 301-331.
22. Weir G C, Clore E T, Zmachinski C J, Bonner-Weir S (1981) Islet secretion in a new experimental model for non-insulin-dependent diabetes. Diabetes 30: 590-595.
23. Velander P, Theopold C, Hirsch T, Bleiziffer O, Zuhaili B, et al. (2008) Impaired wound healing in an acute pig model and the effects of local hyperglycemia. Wound Repair Regen 16:288-293.
24. Hirsch T, Spielmann M, Velander P, Zuhaili B, Bleiziffer O, et al. (2008) Insulin-like growth factor-1 gene therapy and cell transplantation in diabetic wounds. J Gene Med 10: 1247-1252.
25. Hirsch T, Spielmann M, Zuhaili B, Fossum M, Metzig M, et al. (2009) Human beta-defensin-3 promotes wound healing in infected diabetic wounds. The Gene Med 3: 220-228.
26. Velander P, Theopold C, Bleiziffer O, Bergmann J, Svensson H, et al. (2009) Cell suspensions of autologous keratinocytes or autologous fibroblasts accelerate the healing of full thickness skin wounds in a diabetic porcine wound healing model. J Surgical Res 157: 14-20.
27. Bergmann J, Hackl F, Koyama T, Aflaki P, Smith C A, et al. (2009) The effect of amnion-derived cellular cytokine solution on the epithelialization of partial-thickness donor site wounds in normal and Streptozotocin-induced diabetic swine. Eplasty 9: e49.
28. Singer A J, Taira B R, McClain S A, Rooney J, Steinhauff N, et al. (2009) Healing of mid-dermal burns in a diabetic porcine model. J Burn Care Res 30: 880-6.
29. Hackl F, Bergmann J, Granter S R, Koyama T, Kiwanuka E, et al. (2012) Epidermal regeneration by micrograft transplantation with immediate 100-fold expansion. Plast Reconstr Surg 129: 443e-452e.
30. Woodley D T, Fan J, Cheng C F, Li Y, Chen M et al. (2009). Participation of the lipoprotein receptor LRP1 in hypoxia-HSP90αalpha autocrine signaling to promote keratinocyte migration. J Cell Sci 122: 1495-1498.
31. Botusan I R, Sunkari V G, Savu O, Catrina A I, Grünler J, et al. (2008) Stabilization of HIF-1αalpha is critical to improve wound healing in diabetic mice. Proc Natl Acad Sci USA 105: 19426-19431.
32. Catrina S B, Okamoto K, Pereira T, Brismar K, Poellinger L (2004) Hyperglycemia regulates hypoxia-inducible factor-lalpha protein stability and function. Diabetes 53: 3226-3232.
33. Fadini G P, Sartore S, Schiavon M, Albiero M, Baesso I, et al. (2006) Diabetes impairs progenitor cell mobilisation after hindlimb ischaemia-reperfusion injury in rats. Diabetologia 49:3075-3084
34. Gao W, Ferguson G, Connell P, Walshe T, Murphy R, et al. (2007) High glucose concentrations alter hypoxia-induced control of vascular smooth muscle cell growth via a HIF-1alpha-dependent pathway. J Mol Cell Cardiol 42: 609-619.
35. Stephens P, Caley M, Peake M (2013) Alternatives for animal wound model systems. Methods Mol Biol 1037: 177-201.
36. Li W, Fan J, Chen M, Guan S, Sawcer D, et al. (2004) Mechanism of human dermal fibroblast migration driven by type I collagen and platelet-derived growth factor-BB. Mol Biol Cell 15: 294-309.
37. Balaji S, LeSaint M, Bhattacharya S S, Moles C, Dhamija Y, et al. (2014) Adenoviral-mediated gene transfer of insulin-like growth factor 1 enhances wound healing and induces angiogenesis. J Surg Res 190: 367-377.
38. Bevan S, Woodward B, Ng R L H, Green C, Martin R (1997) Retroviral gene transfer into porcine keratinocytes following improved methods of cultivation. Burns 23: 525-532.
39. Woodley D T, Remington J, Huang Y, Hou Y, Li W, et al. (2007) Intravenously injected human fibroblasts home to skin wounds, deliver type VII collagen, and promote wound healing. Mol Ther 15: 628-35.
40. Tsen F, Bhatia A, O'Brien K, Cheng C F, Chen M, et al. (2013) Extracellular heat shock protein 90 signals through subdomain II and the NPVY motif of LRP-1 receptor to Akt1 and Akt2: a circuit essential for promoting skin cell migration in vitro and wound healing in vivo. Mol Cell Biol 33: 4947-59.
41. Kratz, F. (2008) Albumin as a drug carrier: Design of prodrugs, drug conjugates and nanoparticles. J. Control. Release, 132,171-183.
42. Jayaprakash P, Dong H, Zou M, Bhatia A, O'Brien K, et al., and Li W. (2015) Hsp90α and Hsp90β together operate a hypoxia and nutrient paucity stress-response mechanism during wound healing. J. Cell Scie. 128:1475-80.
43. Li, W., D. Sahu & F. Tsen., 2012. Secreted heat shock protein-90 Hsp90 in wound healing and cancer. Biochim Biophys Acta, 1823, 730-41.
44. Sahu, D., Z. Zhao, F. Tsen, C. F. Cheng, R. Park, A. J. Situ, J. Dai, A. Eginli, S. Shams, M. Chen, T. S. Ulmer, P. Conti, D. T. Woodley & W. Li., (2012) A potentially common peptide target in secreted heat shock protein-90CE± for hypoxia-inducible factor-10E±-positive tumors. Mol Biol Cell, 23, 602-13.

What is claimed is:

1. A recombinant polypeptide, wherein the recombinant polypeptide comprises a polypeptide from the group of: an Hsp90α polypeptide sequence (SEQ ID NO.1), an F-8 polypeptide sequence (SEQ ID NO. 2), an F-5 polypeptide sequence (SEQ ID NO. 3), an F-6 polypeptide sequence (SEQ ID NO. 4), and an equivalent of each thereof, wherein the recombinant polypeptide is operatively linked to a non-immunogenic carrier protein.

2. The recombinant polypeptide of claim 1, wherein the polypeptide consists of a polypeptide from the group of: an Hsp90α polypeptide sequence (SEQ ID NO.1), an F-8 polypeptide sequence (SEQ ID NO. 2), an F-5 polypeptide sequence (SEQ ID NO. 3), an F-6 polypeptide sequence (SEQ ID NO. 4), or an equivalent of each thereof.

3. The recombinant polypeptide of claim 1, wherein the equivalent comprises a polypeptide having at least 70% amino acid identity to SEQ ID NOs. 1, 2, 3, or 4 or a polypeptide that hybridizes to a polypeptide encoded by a polynucleotide that hybridizes under conditions of high stringency to a reference polynucleotide encoding a polypeptide comprising SEQ ID NOs. 1, 2, 3 or 4, or the complement of the reference polynucleotide.

4. The recombinant polypeptide of claim 1, wherein the non-immunogenic carrier protein is selected from the group of albumin, pro-albumin, polyethylene glycol (PEG), glutathione S-transferase, thyroglobulin, or keyhole limpet hemocyanin.

5. The recombinant polypeptide of claim 1, wherein the non-immunogenic carrier protein comprises albumin.

6. A polynucleotide, wherein the polynucleotide encodes the recombinant polypeptide of claim 1.

7. A vector comprising the polynucleotide of claim 6.

8. A host cell comprising the polynucleotide of claim 6.

9. A host cell comprising the vector of claim 7.

10. The host cell of claim 9, wherein the host cell is a prokaryotic or a eukaryotic cell.

11. A polypeptide, wherein the polypeptide is produced by the host cell of claim 8 and wherein the polypeptide is optionally isolated.

12. A composition, wherein the composition comprises the recombinant polypeptide of claim 1, and a carrier.

13. The composition of claim 12 further comprising a therapeutic agent other than the recombinant polypeptide.

14. The composition of claim 13, wherein the therapeutic agent is platelet-derived growth factor (PDGF).

15. The composition of claim 12, wherein the composition is formulated for topical administration.

16. A method to promote epidermal tissue regeneration and/or re-epithelialization or prevent cell apoptosis in wounded epithelial tissue, in a subject in need thereof comprising administering to a tissue in need thereof an effective amount of the recombinant polypeptide of claim 1.

17. A method to facilitate healing or treat a wound or an injury, comprising administering to a wounded or injured tissue in a subject in need thereof an effective amount of the recombinant polypeptide of claim 1.

18. The method of claim 17, further comprising administering an effective amount of a wound-healing therapeutic agent other than the recombinant polypeptide.

19. The method of claim 18, wherein the administration of the recombinant polypeptide and the therapeutic agent is concurrent or sequential.

20. The method of claim 17, wherein the subject is a mammal.

21. The method of claim 17, wherein the recombinant polypeptide is administered about every 6 to about every 72 hours or about every 24 to about 48 hours.

22. The method of claim 17, wherein the wound is a skin wound or a wound to an eye.

23. The method of claim 22, wherein the skin wound is an acute wound, a diabetic wound, or a burn wound.

24. The method of claim 23, wherein the acute wound comprises a traumatic wound or a surgical wound.

25. The method of claim 23, further comprising treating or preventing progression or conversion of the burn wound.

26. The method of claim 17, wherein the injury is an eye disease or an eye injury.

27. The method of claim 26, wherein the eye injury is a corneal injury or a conjunctival injury.

28. The method of claim 26, wherein the eye injury is caused by a penetrating object, a foreign body, a chemical, or burn.

29. The method of claim 16, wherein the subject is a mammal.

30. The method of claim 16, wherein the recombinant polypeptide is administered about every 6 to about every 72 hours or about every 24 to about 48 hours.
